(19) United States
(12) Patent Application Publication
(10) Pub. No.: US 2022/0047490 A1
(43) Pub. Date: Feb. 17, 2022

(54) COMPOSITION COMPRISING A POLYSACCHARIDE, A POLYOL AND SPECIFIC ESTER AND OIL

(71) Applicant: L'OREAL, Paris (FR)

(72) Inventors: Emmanuelle PORTOIS, Chevilly La Rue (FR); Pamella WANG, Chevilly La Rue (FR)

(21) Appl. No.: 17/413,168

(22) PCT Filed: Dec. 20, 2019
(86) PCT No.: PCT/EP2019/086734

§ 371 Date: Jun. 11, 2021

(30) Foreign Application Priority Data
Dec. 20, 2018 (FR) 1873638

(51) Int. Cl. A61K 8/60; A61K 8/73; A61K 8/37; A61K 8/34; A61Q 19/10

(52) U.S. Cl. CPC A61K 8/602; A61K 8/73; A61Q 19/10; A61K 8/342; A61K 8/37; A61K 8/375

(57) ABSTRACT

The present invention relates to a composition, preferably a cosmetic composition, comprising at least one polysaccharide comprising rhamnose: at least one polyol, at least one fatty acid and polyglycerol ester comprising 5 to 9 glycerol patterns, and at least 10% by weight of at least one non-volatile hydrocarbon oil relative to the total weight of the composition, chosen from among C₁₀-C₂₆ monoalcohols; monoesters with formula R₁COOR₂ wherein R₂ represents the remainder of a saturated or unsaturated, linear or branched or aromatic fatty acid comprising 4 to 40 carbon atoms, and R₂ represents a hydrocarbon Chain, particularly branched, containing 3 to 40 carbon atoms, provided that R₁+R₂ is greater than or equal to 17; the diesters comprising between 17 and 40 carbon atoms in total, and in particular diesters with formula R′₁COOR′₂OOCR′₃ wherein R′₁ and R′₃ each represent the remainder of a linear N or branched fatty acid comprising from 4 to 15 carbon atoms, and R′₂ represents a hydrocarbon chain, particularly linear, containing 2 to 10 carbon atoms; plant hydrocarbon oils; linear or branched alkanes having 15 to 19 carbon atoms; and mixtures thereof.

This invention relates to a composition, preferably cosmetic, comprising a polysaccharide comprising rhamnose, a polyol, a specific ester and at least 10% by weight of a particular oil.

The skin is a tissue in which cells are contiguous and firmly attached to each other. Skin tissue forms an external coating comprising sebaceous or sudoriferous glands, and hair follicles. The skin, and particularly the scalp, are continuously renewed epithelia.

Renewal, or desquamation, is a coordinated and finely regulated process leading to the elimination of surface cells, insensibly and invisibly.

The human skin is composed of two compartments, namely a surface compartment (the epidermis) and a deep compartment (the dermis).

The epidermis is conventionally divided into a base layer of keratinocytes forming a germinative layer of the epidermis, a layer called the spinous layer composed of several layers of polyhedric cells arranged on the germinative layers, one to three layers called the stratum granulosum composed of flattened cells containing distinct cytoplasmic inclusions, the keratohyalin grains and finally, a set of upper layers called corneal layers (or stratum corneum), composed of keratinocytes at the terminal stage of their differentiation called corneocytes.

Corneocytes are anucleate cells composed principally of a fibrous material containing cytokeratins, surrounded by a corneal envelope. There is permanent production of new keratinocytes to compensate for the continuous loss of epidermal cells in the stratum corneum according to a mechanism called desquamation.

However, an unbalance between the production of cells in the base layer and the desquamation rate can lead to the formation of scales on the skin surface. Similarly, for various reasons, a deficit of terminal differentiation of cells in the stratum corneum can lead to the formation of large, thick clumps of cells, visible to the naked eye and called “dander”, or in other situations, to thinning of the stratum corneum. This can cause fragility of the barrier properties of the epidermis, chronic dehydration of the stratum corneum, loss of mechanical elasticity, tightness, and make the skin lose its luster and transparency. Among examples of factors conducive to this alteration of the surface quality of the skin, mention may be made of stress, the winter period, excess sebum, a hydration disorder; this can also be the case for dry skin of elderly subjects.

Thus, fragility of the skin barrier can occur in the presence of external aggression such as irritants (detergents, acids, bases, oxidants, reducing agents, concentrated solvents, noxious gases or fumes), mechanical actions (friction, shocks, abrasion, surface tearing, projection of dust, particles, shaving or epilation), thermal or climatic unbalances (cold, dryness, radiation), xenobiotic unbalances (undesirable micro-organisms, allergens) or internal aggressions of the psychological stress type.

One of the critical steps in the terminal differentiation process of the stratum corneum is cross-linking of proteic precursors of the cornified envelope. This phenomenon plays an essential role in the development and maintenance of skin cohesion and physical properties of the skin such as the barrier function.

The cornified envelope is an essential component of corneocytes.

Maturing of the cornified envelope from the deep layers to surface layers of the stratum corneum can be characterized by morphological and biophysical or mechanical parameters.

Hydrating agents conventionally used such as moisturizers, hydrating polymers or fatty bodies such as petroleum jelly, temporarily modify the surface properties of the skin. These active agents can increase the mechanical suppleness of the stratum corneum, increase its state of hydration and/or improve the microrelief of the skin by the formation of a surface film on the skin. In general, these effects are not remanent in time and only last for a few hours. Furthermore, after the skin has been cleaned, these active agents are eliminated and the effect of increased mechanical suppleness of the skin, improved skin texture or optical properties of the skin disappear.

Furthermore, the use of-film forming agents on the skin, and particularly the use of moisturizing polysaccharides such as carrageenan, often lead to a skin “tightening” effect, an increase in the elastic modulus of the skin; this increased surface stiffness causes discomfort of the skin. Firstly, it is not unusual that their presence in compositions makes them more tacky and often more difficult to apply. Furthermore, this feeling of discomfort often persists after the composition has dried because once dry, the resulting deposit can leave a relatively rigid film creating an impression of tightness, dryness and sometimes cause a masking effect on the skin.

Therefore there is a need for active agents that improve the state of hydration of the skin, particularly dry skin or old skin, avoiding tightness and the feeling of discomfort during application on the skin. There is also a need for compositions that leave a non-tacky deposit, particularly when drying.

There is also a need for compositions that confer a plumping effect and/or a bouncy appearance on the skin. “Bouncy appearance” means an effect of remodeling the skin. The skin is smoother and has a more fleshy appearance, that remains even after pressing on the skin with a finger.

The inventors have now discovered that the association of a particular polysaccharide, i.e. including rhamnose, with a polyol, an ester of fatty acid and polyglycerol comprising 5 to 9 glycerol patterns and at least 10% by weight of a particular oil, can satisfactorily make the skin more supple, while having a non-tacky deposit. In particular, the association of a particular polysaccharide, i.e. including rhamnose, with a polyol and an ester of fatty acid and polyglycerol comprising 5 to 9 glycerol patterns, has very advantageous effects of making the skin more supple, but results in a deposit that sticks to the skin. The addition of at least 10% by weight of a particular oil, as presented below, very strongly reduces the tackiness. Furthermore, the proposed compositions according to the invention confer a plumping effect and a bouncy appearance on the skin.

Thus, the purpose of the present invention is a composition comprising the following in a physiologically acceptable medium:

at least one polysaccharide containing rhamnose,

at least one polyol,

at least one ester of fatty acid and polyglycerol comprising 5 to 9 glycerol patterns, and

at least 10% by weight relative to the total weight of composition, of at least one non-volatile hydrocarbon oil chosen from among:
C₁₀-C₂₆ monoalcohols,
monoesters with formula R₁COOR₂ wherein R₁ is the remainder of a linear or branched or aromatic fatty acid comprising from 4 to 40 saturated or unsaturated carbon atoms and R₂ is a hydrocarbon chain in particular branched containing from 3 to 40 carbon atoms, with the condition that R₁+R₂ is greater than or equal to 17;
the diesters comprising between 17 and 40 carbon atoms in total, and in particular diesters with formula R′₁COOR′₂OOCR′₃ wherein R′₁ and R′₃ each represent the remainder of a linear or branched (preferably linear) fatty acid comprising from 4 to 15 carbon atoms, and R′₂ represents a hydrocarbon chain, particularly linear, containing 2 to 10 carbon atoms;
vegetable hydrocarbon oils;
linear or branched alkanes having from 15 to 19 carbon atoms; and
mixtures thereof.

The composition according to the invention is preferably cosmetic.

“Physiologically acceptable” means a medium compatible with keratin materials.

Another purpose of this invention is a method of cosmetic treatment of keratin fibers, preferably the skin, comprising application of a composition according to the invention on said keratin fibers.

Another purpose of this invention is cosmetic use of a composition according to the invention to make the skin more supple, particularly the stratum corneum.

Another purpose of the invention is a composition preferably cosmetic, comprising, in a physiologically acceptable medium:

at least one polysaccharide comprising rhamnose,

at least at least one hydrophilic active agent, preferably chosen from the C-glycoside

derivatives of general formula (F) below:

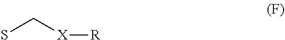

in which:
R denotes an unsubstituted linear C1-C4 alkyl radical, especially C1-C2, in particular methyl;
   S represents a monosaccharide chosen from D-glucose, D-xylose, N-acetyl-D-glucosamine or L-fucose, and in particular D-xylose;
   X represents a group chosen from —CO—, —CH(OH)—, —CH(NH2)-, and preferentially a —CH(OH)— group;

as well as their cosmetically acceptable salts, their solvates such as hydrates and their optical isomers; and preferably chosen from C-beta-D-xylopyranoside-2-hydroxy-propane and C-alpha-D-xylopyranoside-2-hydroxy-propane, and

optionally at least 10% by weight relative to the total weight of composition, of at least one non-volatile hydrocarbon oil chosen from among:
C₁₀-C₂₆ monoalcohols,
monoesters with formula R₁COOR₂ wherein R₁ is the remainder of a linear or branched or aromatic fatty acid comprising from 4 to 40 saturated or unsaturated carbon atoms and R₂ is a hydrocarbon chain in particular branched containing from 3 to 40 carbon atoms, with the condition that R₁+R₂ is greater than or equal to 17;
the diesters comprising between 17 and 40 carbon atoms in total, and in particular diesters with formula R′₁COOR′₂OOCR′₃ wherein R′₁ and R′₃ each represent the remainder of a linear or branched (preferably linear) fatty acid comprising from 4 to 15 carbon atoms, and R′₂ represents a hydrocarbon chain, particularly linear, containing 2 to 10 carbon atoms;
vegetable hydrocarbon oils;
linear or branched alkanes having from 15 to 19 carbon atoms; and
mixtures thereof.

Polysaccharide

The composition according to the invention comprises at least one polysaccharide comprising rhamnose.

Preferably, the polysaccharide according to the invention comprises rhamnose amount varying from 10% to 100% by weight relative to the total weight of polysaccharide, preferably from 20% to 70% by weight, and more preferably from 40% to 60% by weight.

Preferably, the polysaccharide according to the invention is not sulfated. “Not sulfated” means that the sulfation ratio of the polysaccharide is less than 0.5% by weight, preferably less than 0.1% by weight relative to the weight of polysaccharide. Preferably, the sulfation ratio is zero.

Thus preferably, the polysaccharide according to the invention is such that the repetitive elements from which it is composed contain predominantly rhamnose.

Preferably, the repetitive elements comprise at least components with general formula I:

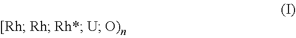

wherein Rh is a rhamnose molecule, Rh* is a rhamnose molecule fixed in a branched manner, O is a molecule of a hexosidic or pentosidic sugar, U is a molecule of uronic acid and n is between 1 and 100, and preferably between 5 and 65.

“Repetitive elements containing predominantly rhamnose” means a branched chain comprising at least 50% of series D and/or L rhamnose, and its α and/or β isomers.

The sugar O may in particular be chosen from among fucose, galactose, ribose, arabinose, xylose and mannose.

Uronic acid U means any hexose oxidized on its primary alcohol function into carboxylic acid, and particularly glucuronic acid, galacturonic acid, mannuronic acid or iduronic acid.

According to one particular embodiment of the invention, the branched rhamnose molecule can be fixed by an osidic bond from its carbon 1 on a free carbon of one among the sugar molecule O or uronic acid molecule U or rhamnose molecule Rh of the saccharidic chain, particularly on carbons 2 or 3.

According to another particular embodiment, the repetitive elements can be composed in particular by the sequence with general formula II:

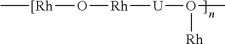

wherein Rh is a rhamnose molecule, O is a hexosidic or pentosidic sugar molecule, U is a uronic acid molecule and the rhamnose branch onto the ose O consists of an osidic bond (1→2) or (1→3).

According to one particularly interesting embodiment, the sugar O is galactose and the uronic acid U is glucuronic acid. Preferably, the sequence has a chain containing 3 rhamnose molecules, one of which is branched, 2 galactose molecules and one glucuronic acid molecule. According to formula II, n represents value such that this polysaccharide has a molecular weight of the order of 50000 daltons. It can be obtained from Klebsiella type bacteria cultures, particularly Klebsiella pneumoniae and particularly the 1-714 strain (deposed at CNCM—Collection Nationale de Culture de Microorganismes (National Microorganisms Culture Collection) as number 1-714) according to a method described below. Advantageously, this polysaccharide has the rhamnose branch on galactose in position V. It is found that this polysaccharide is composed particularly of the following repetitive unit: →4)-α-L-Rhap(1→3)-β-D-Galp(1→2)-α-L-Rhap(1-4)-β-D-GlcpA(1→3)-[α-L-Rhap(1→2)]-α-D-Galp(1→.

Hydrolysis of this polysaccharide also makes it possible to obtain a mixture of fractions with a lower molecular weight, particularly majority fractions of 5 000 daltons and 13 000 daltons, possibly purifiable and particularly interesting according to the invention.

According to another particular embodiment, the repetitive elements can be composed in particular by the sequence with general formula III:

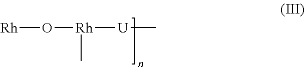

wherein Rh is a rhamnose molecule, 0 is a hexosidic or pentosidic sugar molecule, U is a uronic acid molecule and rhamnose is branched onto the rhamnose by an osidic bond (1-3). According to one particularly interesting embodiment, the sugar O is glucose and the uronic acid U is glucuronic acid, preferably a chain containing 3 rhamnose molecules including one branched molecule, one glucose molecule and one glucuronic acid molecule. Such a polysaccharide can be obtained in particular according to the method described below from a culture of Klebsiella planticola type bacteria, particularly the I-2743 strain (deposited at CNCM as number 1-2743). Advantageously, such a polysaccharide has the rhamnose branch on the rhamnose in position III. It is found that this polysaccharide is composed more particularly of the following repetitive unit: →3)-β-L-Rhap(1→4)-β-D-Glcp(1→2)-[α-L-Rhap(1→3)]-α-L-Rhap(1→4)-α-D-GlcpA(1→.

Hydrolysis of this polysaccharide also makes it possible to obtain a mixture of fractions with a lower molecular weight, particularly the majority fraction of 5 000 daltons, possibly purifiable and particularly interesting according to the invention.

In general, the polysaccharides according to the invention can be of bacterial or vegetable origin. They can be obtained by classical polysaccharide production techniques (chemical synthesis, enzymatic extraction from exopolysaccharides). According to one advantageous embodiment, the polysaccharides are exopolysaccharides obtained by fermentation of a bacterial strain producing them, of the encapsulated bacteria type, according to a production method like that described in detail in patent FR264522. This method is defined in that a Klebsiella type bacteria strain is put into culture in a nutrient medium comprising a carbon source, a preferential nitrogen source and appropriate mineral salts, at a pH of about 6 to 8, at a temperature of about 30 to 35° C., while stirring and under aeration, for 4 to 12 days. The carbon/nitrogen ratio is advantageously more than 5 so as to favor secretion of the polysaccharide. The polysaccharide can then be isolated by submitting the fermentation medium to heat treatment at about 70-120° C. for about 10 minutes to 1 hour, then by separating it, for example by centrifuging it cold. The exopolysaccharides and cellular polysaccharides are all contained in the clear float phase. If necessary, the polysaccharides can be purified by precipitation by the addition of a non-solvent organic liquid such as acetone or a lower alcohol such as ethanol or propanol, and separated by filtration or centrifuging before being dried.

The isolated polysaccharides can thus be easily incorporated into a composition, as is or in hydrolyzed form. In this case, the hydrolysis can be done before drying using known methods such as acid hydrolysis. It can be done using a frequently used proton donor such as hydrochloric acid, at a temperature varying between 50 and 100° C. for between 30 minutes and 4 hours, depending on the required size of the fractions. The oligosaccharidic fractions thus obtained can be recovered and purified if necessary, using classical methods.

This protocol can be done using bacterial strains producing exopolysaccharides rich in rhamnose, and particularly encapsulated bacteria. According to one preferred embodiment of the invention, a strain of Klebsiella bacteria will be used, preferably Klebsiella pneumoniae or Klebsiella planticola.

Preferably, the repetitive unit of the polysaccharide (exopolysaccharide) according to the invention is that produced by Klebsiella pneumoniae 1-714 and called Rhamnosoft®:

The composition of Rhamnosoft® corresponds to a polymer with a branched structure, with a molecular weight of the order of 50 000 daltons, and having a saccharidic sequence comprising three molecules of rhamnose (1, Ill, VI), two molecules of galactose (11, V) and one molecule of glucuronic acid (IV). Therefore rhamnose makes up 50% of the polysaccharide. The polysaccharide has a rhamnose VI branch on the galactose in position V.

In this case the structure of the repetition unit is:

→4)-α-L-Rhap(1→3)-β-D-Galp(1→2)-α-L-Rhap(1→4)-β-D-GlcpA(1→3)-[α-L-Rhap(1→2)]-α-D-Galp(1→.

It corresponds to the following detailed formula:

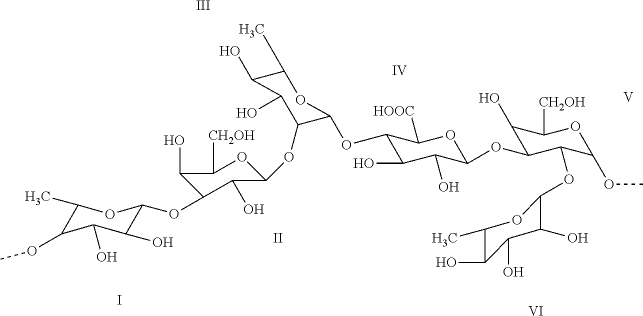

Preferably, the polysaccharide according to the invention is used in an aqueous solution at 2.5% by weight of active material, relative to the total weight of the solution. Such a polysaccharide is marketed particularly under the name Rhamnosoft® HP 1.5P by Solabia.

Preferably, the polysaccharide may be present in the composition according to the invention with a dry matter content ranging from 0.01% to 1% by weight relative to the total weight of the composition, preferably from 0.05% to 0.5% by weight, and more preferably from 0.1% to 0.3% by weight.

Ester of fatty acid and polyglycerol comprising 5 to 9 glycerol patterns

The composition according to the invention also comprises at least one ester of fatty acid and of polyglycerol comprising 5 to 9 carbon atoms.

The ester of fatty acid and polyglycerol is formed from at least one acid comprising an alkyl or alkenyl chain containing from 12 to 20 carbon atoms and 5 to 9 glycerol patterns, preferably from 5 to 6 glycerol patterns.

According to one embodiment, the polyglycerol ester according to the invention results from esterification of at least one saturated or unsaturated fatty acid and a polyglycerol.

Preferably, the ester of fatty acid and polyglycerol comprising from 5 to 9 glycerol patterns is a mono- or diester, and preferably a mono-ester.

The term “polyglycerol” designates glyceryl polymers that are linear chains of 5 to 9, and preferably 5 to 6 glycerol units.

The esters considered most particular for the purposes of this invention are esters resulting from esterification of polyglycerol and C12-20, preferably C12 to C18, and more preferably C12 carboxylic acid(s), such as lauric, oleic, stearic, isostearic or myristic acids.

The carboxylic acid may be linear or branched, saturated or unsaturated.

Preferably, it is a linear monocarboxylic acid.

In general, they are derived from esterification of at least one hydroxyl function of a polyglycerol by a C12-C20, preferably C12 to C18, and more particularly C6 to C18, and particularly C10 to C12 carboxylic acid.

According to one particular embodiment, esters suitable for this invention can be derived from esterification of a polyglycerol by one or several identical or different carboxylic acids. It may be a hydroxylated mono-ester, a hydroxylated di-ester, a hydroxylated tri-ester, or a mixture thereof.

In one preferred embodiment of the invention, the fatty acid and polyglycerol ester is chosen from among polyglyceryl monolaurate comprising 5 to 6 glycerol patterns, polyglyceryl monooleate comprising from 5 to 6 glycerol patterns, polyglyceryl mono(iso)stearate comprising 5 to 6 glycerol patterns, polyglyceryl dioleate comprising 5 to 6 glycerol patterns, polyglyceryl monomyristate comprising 5 to 6 glycerol patterns, and mixtures thereof.

In another preferred embodiment of the invention, the fatty acid and polyglycerol ester has an HLB (Hydrophilic Lipophilic Balance) value equal to between 10 and 13.

Advantageously, the composition according to the invention comprises a fatty acid and polyglycerol ester that is a polyglyceryl monolaurate with 5 to 6 glycerol patterns, i.e. polyglyceryl-5 laurate or polyglyceryl-6 laurate.

A commercial project predominantly based on polyglyceryl-5 laurate or PG-5 laurate is available under the tradename SUNSOFT A-121 E-C® by Taiyo Kagaku.

A commercial project predominantly based on polyglyceryl-6 laurate or PG-6 laurate is available under the tradename Dermofeel G6L by Dr Straetmans.

The fatty acid and polyglycerol ester comprising 5 to 9 glycerol patterns may be present in the composition according to the invention in a content of at least 1% by weight, preferably ranging from 1% to 10% by weight relative to the total weight of the composition, preferably from 3% to 7% by weight, and more preferably from 4% to 6% by weight.

Polyol

The composition according to the invention also comprises at least one polyol.

For the purposes of the invention, polyol means a hydrocarbon chain comprising at least 2 carbon atoms, preferably 2 to 50 carbon atoms, preferably 4 to 20 carbon atoms, preferably having 2 to 10 carbon atoms, and preferably having 2 to 6 carbon atoms, and carrying at least 2 hydroxy groups. The polyols used in this invention can have an average molecular mass by weight of less than or equal to 1000, and preferably between 90 and 500.

The polyol may be a natural or synthetic polyol. The polyol can have a linear, branched or cyclic molecular structure.

This polyol can be chosen from among glycerine and its derivatives, and glycols and their derivatives. The polyol can be chosen from the group composed of glycerine, diglycerine, polyglycerine, diethylene glycol, propylene glycol, dipropylene glycol, butylene glycol, pentylene glycol, hexylene glycol, 1,3-propanediol, 1,5-pentanediol, octane 1,2-diol, polyethyleneglycols, particularly with 5 to 50 ethylene oxide groups, and sugars such as sorbitol, and mixtures thereof.

More particularly, the polyol is glycerine.

Said polyol(s) can be present in a quantity ranging from 2% to 30% by weight, relative to the total weight of the composition, preferably ranging from 3% to 25% by weight, and preferably ranging from 5% to 20% by weight.

Oil

The composition according to the invention comprises at least one non-volatile hydrocarbon oil, with a content of at least 10% by weight relative to the total weight of composition. Preferably, the composition comprises non-volatile hydrocarbon oil with a content of 10% to 40%, preferably 10% to 30% by weight relative to the total weight of the composition.

The oil is chosen from among:
C₁₀-C₂₆ monoalcohols,
monoesters with formula R₁COOR₂ wherein R₁ is the remainder of a linear or branched or aromatic fatty acid comprising from 4 to 40 saturated or unsaturated carbon atoms and R₂ is a hydrocarbon chain in particular branched containing from 3 to 40 carbon atoms, with the condition that R₁+R₂ is greater than or equal to 17;
the diesters comprising between 17 and 40 carbon atoms in total, and in particular diesters with formula R′₁COOR′₂OOCR′₃ wherein R′₁ and R′₃ each represent the remainder of a linear or branched (preferably linear) fatty acid comprising from 4 to 15 carbon atoms, and R′₂ represents a hydrocarbon chain, particularly linear, containing 2 to 10 carbon atoms;
vegetable hydrocarbon oils;
linear or branched alkanes having from 15 to 19 carbon atoms; and
mixtures thereof.

“Oil” refers to a non-aqueous compound, liquid at 25° C. at atmospheric pressure (1.013×10⁵ Pa), not water-miscible.

“Not miscible” means that the mixture of the same quantity of water and oil, after stirring, does not lead to a stable solution that comprises only a single phase, in the aforementioned conditions of temperature and pressure. The observation is made with the naked eye or using a phase contrast microscope if necessary, on 100 g of mixture obtained after Rayneri stirring sufficient to cause a vortex to appear within the mixture (for the purposes of information 200 to 1000 rpm); the resulting mixture being left to sit, in a closed bottle, for 24 hours at ambient temperature before observation.

“Non-volatile oil” refers to an oil of which the vapor pressure at 25° C. and atmospheric pressure, is not zero and is less than 10⁻³ mm of Hg (0.13 Pa).

The term “hydrocarbon oil” refers to an oil essentially formed, or consisting, of carbon and hydrogen atoms, and optionally oxygen, nitrogen atoms, and containing no silicon or fluorine. The hydrocarbon oil is therefore separate from a silicone oil and from a fluorine oil.

It may contain alcohol, ester, ether, carboxylic acid, amine and/or amide groups. Preferably, the hydrocarbon oil is free of heteroatoms such as nitrogen, sulfur and phosphorus.

In particular, this non-volatile hydrocarbon oil may comprise at least one alcohol function (it is then an “alcohol oil”) or at least one ester function (it is then an “ester oil”).

The non-volatile hydrocarbon oil according to the invention is chosen from among:
C₁₀-C₂₆ alcohols, preferably monoalcohols;

More particularly, the C₁₀-C₂₆ alcohols are saturated or not, branched or not, and comprise from 10 to 26 carbon atoms.

Preferably, the C₁₀-C₂₆ alcohols are fatty alcohols, preferably branched when they contain at least 16 carbon atoms.

As examples of fatty alcohols that can be used according to the invention, mention can be made of linear or branched fatty alcohols with synthetic origin, or natural such as for example alcohols coming from plant substances (coconut, palm kernel, palm, etc.) or animal substances (tallow, etc.).

Of course, other long-chain alcohols can also be used, such as for example ether-alcohols or so-called Guerbet alcohols.

Finally, certain more or less long cuts of alcohols of natural origin, such as for example coco (C₁₂ to C₁₆) or tallow (C₁₆ to C₁₈) or compounds of the diol or cholesterol type, can also be used.

Preferably a fatty alcohol comprising from 10 to 24 carbon atoms, and more preferably from 12 to 22 carbon atoms is used.

As particular examples of fatty alcohols that can be used preferably, mention can be made in particular of lauric, isostearyl, oleic alcohol, 2-butyloctanol, 2-undecyl pentadecanol, 2-hexyldecylic alcohol, isocetylic alcohol, octyldodecanol and mixtures thereof.

According to one advantageous embodiment of the invention, the alcohol is octyldodecanol. Preferably, the content of octyldodecanol is between 10% and 30% by weight of the total weight of the composition, preferably between 10% and 20% by weight.
the ester oils having between 17 and 70 carbon atoms chosen from among;
   monoesters comprising between 17 and 40 carbon atoms in total, in particular monoesters, having formula R₁COOR₂ wherein R₁ is the remainder of a linear or branched or aromatic fatty acid comprising from 4 to 40 carbon atoms, saturated or not, and R₂ is a hydrocarbon chain in particular branched containing from 3 to 40 carbon atoms with the condition that R₁+R₂ is greater than or equal to 17, as for example Purcellin oil (cetostearyl octanoate), isononyl isononanoate, C₁₂ to C₁₅ alcohol benzoate, 2-ethyl hexyl palmitate, octyldodecyl neopentanoate, octyl-2-dodecyl stearate, octyl-2-dodecyl erucate, isostearyl isostearate, octyl-2 dodecyl benzoate, octanoates, decanoates or ricinoleates of alcohols or of polyalcohols, isopropyl myristate, isopropyl palmitate, butyl stearate, hexyl laurate, 2-ethyl-hexyl palmitate, 2-hexyl-decyl laurate, 2-octyl-decyl palmitate, 2-octyldodecyl myristate. Preferably, the monoester is different from isostearyl neopentanoate.

Preferably, these are esters having formula R₁COOR₂ wherein R₁ is the remainder of a linear or branched fatty acid comprising from 4 to 40 carbon atoms and R₂ is a hydrocarbon chain in particular branched containing from 3 to 40 carbon atoms, with R₁ and R₂ being such that R₁+R₂ is greater than or equal to 17. Preferably, the monoester is different from isostearyl neopentanoate

More particularly, the ester comprises between 17 and 40 carbon atoms in total.

As preferred monoesters, mention can be made of isononyl isononanoate and/or isopropyl myristate. Preferably, the content of isononyl isononanoate present is between 5% and 30% by weight relative to the total weight of the composition. Preferably, the content of isopropyl myristate present is between 5% and 20% by weight relative to the total weight of the composition.
the diesters comprising between 17 and 40 carbon atoms in total, and in particular diesters with formula R′₁COOR′₂OOCR′₃ wherein R′₁ and R′₃ each represent the remainder of a linear or branched fatty acid (preferably linear) comprising from 4 to 15 carbon atoms, and R′₂ represents a hydrocarbon chain, particularly linear, containing 2 to 10 carbon atoms. Preferably R′₁ and R′₃ are identical. As an example, mention can be made of 1,3-propanediol dicaprylate. Such a diester is marketed particularly under the name DUB Zenoat by STEARINERIES DUBOIS. Preferably, the content of 1,3-propanediol dicaprylate present is between 51% and 10% by weight relative to the total weight of the composition.
plant-based hydrocarbon oils such as fatty acid liquid triglycerides (liquid at ambient temperature), in particular fatty acids having from 7 to 40 carbon atoms, such as heptanoic or octanoic acid triglycerides or jojoba oil; in particular, mention can be made of saturated triglycerides such as caprylic/capric triglyceride and mixtures thereof, for example such as the one sold under the reference Myritol 318 by Cognis, glycerol triheptanoate, glycerine trioctanoate, C₁₈₋₃₆ acid triglycerides such as those sold under the reference DUB TGI 24 by Stearineries Dubois), and unsaturated triglycerides such as castor oil, olive oil, ximenia oil or pracaxi oil.
linear or branched alkanes having from 15 to 19 carbon atoms:

As an example of alkanes suitable for the invention, mention may be made of mixtures of alkanes with between 15 and 19 carbon atoms with a molecular weight of between 200 g/mol and 250 g/mol.

Preferably, the alkanes are branched.

Preferably, the alkanes are of plant origin.

Preferably, the mixture used comprises 95 to 99% of C15-C19 branched alkanes by weight relative to the total weight of the mixture, and 1 to 5% by weight of C12-C14 and C20-C26 alkanes relative to the total weight of the mixture. Such a mixture preferably has a molecular weight of about 216 g/mol. Such a mixture is marketed by Seppic under the reference Emogreen L15.

Alternatively and preferably, the mixture used comprises 95 to 99% of the mixture of C15-C19 branched alkanes by weight relative to the total weight of the mixture, and 1 to 5% of C12-C14 and C20-C26 alkanes by weight relative to the total weight of the mixture. Such a mixture preferably has a molecular weight of about 248 g/mol. Such a mixture is marketed by Seppic under the reference Emogreen L19.

Alternatively, a mixture is used comprising 10% by weight of C15-C19 branched alkanes relative to the total weight of the mixture, and 1 to 5% by weight of C12-C14 and C20-C26 alkanes relative to the total weight of the mixture. Such a mixture is marketed by Seppic under the reference Emosmart L19.

Preferably, the content of linear or branched alkane with 15 to 19 carbon atoms is between 10% and 30% by weight relative to the total weight of the composition.
and mixtures thereof.

Preferably, the oil is a branched alkane having from 15 to 19 carbon atoms.

Alternatively and preferably, the oil is a C₁₀-C₂₆ monoalcohol.

Alternatively and preferably, the oil is a mixture between a monoester with formula R₁COOR₂ wherein R₁ represents the remainder of a saturated or unsaturated, linear or branched or aromatic fatty acid comprising 4 to 40 carbon atoms, and R₂ represents a hydrocarbon chain, particularly branched, containing 3 to 40 carbon atoms, provided that R₁+R₂ is greater than or equal to 17, and a diester comprising a total of between 17 and 40 carbon atoms. Preferably, the monoester is different from isostearyl neopentanoate.

Alternatively, and preferably, the oil is a plant hydrocarbon oil such as jojoba oil.

Preferably, the composition according to the invention is an emulsion. Preferably, the composition according to the invention comprises an aqueous phase and an oily phase, said aqueous and oily phases being as defined above. Preferably, the composition according to the invention is an oil-in-water emulsion.

When the composition according to the invention comprises at least one surfactant as described below, it preferably has the aspect of a cream, particularly a white cream.

When the composition according to the invention does not comprise a surfactant as described below, it corresponds to a micro-emulsion.

Surfactants

The composition according to the invention preferably comprises at least one fatty acid and polyethylene glycol ester, as surfactant. Preferably, it also comprises an additional surfactant chosen from among C₁₆-C₂₂ fatty acid and sorbitan esters and C₁₆-C₂₂ fatty acid and glyceryl esters.

The fatty acid and polyethylene glycol ester present in the composition according to the invention is preferably a C₁₆-C₂₂ fatty acid ester comprising 8 to 100 ethylene oxide units. The fatty chain of esters can be chosen particularly among the stearyl, behenyl, arachidyl, palmityl, cetyl patterns and mixtures thereof, such as cetearyl, and preferably a stearyl chain.

The number of ethylene oxide units can vary from 8 to 100, preferably from 10 to 80, and even better from 10 to 50. According to one particular embodiment of the invention, this number can vary from 20 to 40.

As an example of a fatty acid and polyethylene glycol ester, mention may be made of stearic acid esters comprising 20, 30, 40, 50 or 100 units of ethylene oxide, such as products marketed under the tradename Myrj 49 P (polyethylene glycol stearate 20 OE; CTFA name: PEG-20 stearate), Myrj 51, Myrj 52 P (polyethyleneglycol stearate 40 OE; CTFA name: PEG-40 stearate), Myrj 53 or Myrj 59 P by CRODA.

The fatty acid and polyethylene glycol ester may be present in the composition according to the invention in a content ranging from 0.1% to 10% by weight relative to the total weight of the composition, preferably from 0.1% to 5% by weight, and more preferably from 0.25% to 1.5% by weight.

Preferably, the composition according to the invention also comprises an additional emulsifying surfactant chosen from among C₁₆-C₂₂ fatty acid and sorbitan esters and C₁₆-C₂₂ fatty acid and glyceryl esters.

According to a first embodiment of the invention, the composition comprises a C₁₆-C₂₂ fatty acid and sorbitan ester.

The C₁₆-C₂₂ fatty acid and sorbitan esters are formed by esterification of at least one fatty acid comprising at least one saturated or unsaturated linear alkyl chain with 16 to 22 carbon chains, with sorbitol. In particularly, these esters can be chosen from among stearates, behenates, arachidates, palmitates, oleates of sorbitan, and mixtures thereof. Sorbitan stearates and palmitates will be used in preference, and more preferably sorbitan stearates.

The C₁₆-C₂₂ fatty acid and sorbitan ester present in the composition according to the invention is advantageously solid at a temperature of less than or equal to 45° C.

As example of a sorbitan ester that can be used in the composition according to the invention, mention can be made of sorbitan monostearate (CTFA name: Sorbitan stearate) sold by Croda under the tradename Span 60, sorbitan tristearate sold by Croda under the tradename Span 65 V, sorbitan monopalmitate (CTFA name: Sorbitan palmitate) sold by Croda under the tradename Span 40, sorbitan monoleate sold by Croda under the tradename Span 80 V, sorbitan trioleate sold by Uniquema under the tradename Span 85 V, preferably the sorbitan ester used is sorbitan tristearate.

The C₁₆-C₂₂ fatty acid and sorbitan ester can be present in the composition according to the invention in a content ranging from 0.01% to 10% by weight relative to the total weight of the composition, preferably from 0.01% to 5% by weight, and more preferably from 0.25% to 1.5% by weight.

The glyceryl and fatty acid ester can be obtained particularly using an acid comprising a saturated linear alkyl chain, with 16 to 22 carbon atoms. As a glyceryl and fatty acid ester, particular mention may be made of glyceryl stearate (glyceryl mono-, di- and/or tri-stearate) (CTFA name: Glyceryl stearate), glyceryl ricinoleate, and mixtures thereof. Preferably the glyceryl and fatty acid ester used is chosen from among glyceryl stearates.

The glyceryl and fatty acid ester can be present in a quantity ranging from 0.1 to 10% by weight, relative to the total weight of the composition, preferably ranging from 0.1 to 5% by weight, and preferably ranging from 0.5% to 3% by weight.

In particular, the composition according to the invention may comprise a mixture of glyceryl stearate and polyethylene glycol monostearate 100 OE, and in particular that comprising a 50/50 mixture marketed under the tradename Arlacel 165 by Croda.

Aqueous Phase

Preferably, in addition to polyol, the composition according to the invention comprises a physiologically acceptable aqueous medium. “Physiologically acceptable” means a medium compatible with keratin materials.

The composition according to the invention preferably comprises an aqueous medium comprising water and possibly an organic solvent soluble in water, at 25° C., chosen for example among linear or branched C2-C4 alkanols such as ethanol and isopropanol, propanol, butanol; and mixtures thereof.

The composition generally comprises from 10 to 95% by weight of water with respect to the total weight of the composition and preferably from 40 to 80%.

The quantity of organic solvents can range for example from 0 to 30% by weight, preferably from 0.5 to 25% by weight, better from 1 to 20% by weight, even better from 2 to 20% by weight relative to the total weight of the composition.

The composition can also comprise at least one polymer, and particularly a hydrophilic polymer. Such a polymer is preferably a water-soluble or water-dispersible AMPS® polymer.

The water-soluble or water-dispersible AMPS® polymers preferably have a molar mass ranging from 50,000 g/mole to 10,000,000 g/mole, preferably from 80,000 g/mole to 8,000,000 g/mole, and more preferably from 100,000 g/mole to 7,000,000 g/mole.

As water-soluble or water-dispersible AMPS homopolymers suitable for the invention, mention may be made of optionally cross-linked polymers of sodium 2-acrylamido-2-methylpropane sulfonate acid such as that used in the commercial product SIMULGEL 800 (CTFA name: Sodium Polyacryloyldimethyl Taurate), cross-linked polymers of ammonium 2-acrylamido-2-methyl propane sulfonate acid (INCI name: AMMONIUM POLYACRYLDIMEHYLTAURAMIDE) such as the product sold under the tradename HOSTACERIN AMPS@ by Clariant.

As water-soluble or water-dispersible AMPS copolymers according to the invention, mention may be made for example of:
cross-linked sodium acrylamide/acrylamido-2-methyl propane sulfonate copolymers such as those used in the commercial product SEPIGEL 305 (CTFA name: 5 3028758 57 POLYACRYLAMIDE/C13-C14 ISOPARAFFIN/LAURETH-7) or that used in the commercial product sold under the name SIMULGEL 600 (CTFA name: ACRYLAMIDE/SODIUM ACRYLOYLDIMETHYLTAURATE/ISOHEXADECANE/POLYSORBATE-80) by SEPPIC;
copolymers of AMPS® and vinylpyrrolidone or vinylformamide such as that used in the commercial product sold under the tradename ARISTOFLEX AVC® by CLARIANT (CTFA name: AMMONIUM ACRYLOYLDIMETHYLTAURATE/VP COPOLYMER) but neutralized with soda or potash;
copolymers of AMPS® and sodium acrylate, such as for example the AMPS/sodium acrylate copolymer such as that used in the commercial product sold under the tradename SIMULGEL EG® by SEPPIC or under the tradename SEPINOV EM as (CTFA name: HYDROXYETHYL ACRYLATE/SODIUM ACRYLOYLDIMETHYL TAURATE COPOLYMER);
copolymers of AMPS® and hydroxyethyl acrylate, such as for example the AMPS®/hydroxyethyl acrylate copolymer like that used in the commercial product sold under the tradename SIMULGEL NS® by SEPPIC (CTFA name: HYDROXYETHYL ACRYLATE/SODIUM ACRYLOYLDIMETHYLTAURATE COPOLYMER (AND) SQUALANE (AND) POLYSORBATE 60) or as the product marketed under the name SODIUM ACRYLAMIDO-2-M ETHYLPROPAN E SULFONATE/HYDROXYETHYLACRYLATE COPOLYMER such as the commercial product SEPINOV EMT 10 (INCI name: HYDROXYETHYL ACRYLATE/SODIUM ACRYLOYLDIMETHYL TAURATE COPOLYMER);
copolymers of AMPS® and ethoxylated cetearyl methacrylate, possibly cross-linked, such as Aristoflex® HMS, that has the name AMPS copolymer/ethoxylated cetearyl methacrylate (25 EO) 80/20, cross-linked by trimethylolpropane triacrylate (TMPTA) or Ammonium Acryloyldimethyltaurate/Steareth-25 methacrylate crosspolymer with INCI name.

Hydrophilic Active Agent(s)

The composition according to the invention may comprise an aqueous at least one hydrophilic active agent. By “hydrophilic active agent” it is meant an active agent which is hydrosoluble or hydrodispersible, and which is capable of forming hydrogen bonds.

Examples of hydrophilic active agents that may be mentioned include moisturizing agents; depigmenting agents, desquamating agents, anti-aging agents, mattifying agents; healing agents; antibacterial agents; and their mixtures.

Preferably the hydrophilic active agent is chosen from the C-glycoside derivatives of general formula (F) below:

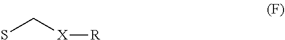

in which:
R denotes an unsubstituted linear C1-C4 alkyl radical, especially C1-C2, in particular methyl;
   S represents a monosaccharide chosen from D-glucose, D-xylose, N-acetyl-D-glucosamine or L-fucose, and in particular D-xylose;
   X represents a group chosen from —CO—, —CH(OH)—, —CH(NH2)-, and preferentially a —CH(OH)— group;
   as well as their cosmetically acceptable salts, their solvates such as hydrates and their optical isomers.

By way of non-limiting illustration of the C-glycoside of formula (F) that is more particularly suitable for the invention, the following compounds may be mentioned:
C-beta-D-xylopyranoside-n-propan-2-one;
C-alpha-D-xylopyranoside-n-propan-2-one;
C-beta-D-xylopyranoside-2-hydroxy-propane;
C-alpha-D-xylopyranoside-2-hydroxy-propane;
1-(C-beta-D-glucopyranosyl)-2-hydroxy-propane;
1-(C-alpha-D-glucopyranosyl)-2-hydroxy-propane;
1-(C-beta-D-glucopyranosyl)-2-amino-propane;
1-(C-alpha-D-glucopyranosyl)-2-amino-propane;
3′-(acetamido-C-beta-D-glucopyranosyl)-propan-2′-one;
3′-(acetamido-C-alpha-D-glucopyranosyl)-propan-2′-one;
1-(acetamido-C-beta-D-glucopyranosyl)-2-hydroxy-propane;
1-(acetamido-C-beta-D-glucopyranosyl)-2-amino-propane;
   as well as their cosmetically acceptable salts, their solvates such as hydrates and their optical isomers.

Preferably, C-beta-D-xylopyranoside-2-hydroxy-propane or C-alpha-D-xylopyranoside-2-hydroxy-propane, and more preferably C-beta-D-xylopyranoside-2-hydroxy-propane, are used. Preferably, a C-glycoside of formula (F) that is suitable for the invention may advantageously be C-beta-D-xylopyranoside-2-hydroxy-propane, whose INCI name is HYDROXYPROPYL TETRAHYDROPYRANTRIOL, sold especially under the name MEXORYL SBB® or MEXORYL SON® by CHIMEX. The salts of C-glycosides of formula (F) suitable for the invention may comprise conventional physiologically acceptable salts of these compounds such as those formed from organic or inorganic acids. By way of example, mention may be made of mineral acid salts, such as sulfuric acid, hydrochloric acid, hydrobromic acid, hydroiodic acid, phosphoric acid and boric acid. Mention may also be made of organic acid salts, which may comprise one or more carboxylic acid, sulphonic acid or phosphonic acid groups. It may be linear, branched or cyclic aliphatic acids or aromatic acids. These acids may furthermore comprise one or more heteroatoms chosen from 0 and N, for example in the form of hydroxyl groups. These include propionic acid, acetic acid, terephthalic acid, citric acid or tartaric acid. Acceptable solvates for the compounds described above include conventional solvates such as those formed in the last step of preparing said compounds due to the presence of solvents. By way of example, mention may be made of solvates due to the presence of water or of linear or branched alcohols, such as ethanol or isopropanol. C-glycosides (I) are known from WO 02/051828.

According to one embodiment, the composition according to the invention comprises a C-glycoside in an amount of between 0.05% and 10% by weight of active ingredient (C-glycoside) relative to the total weight of the composition, in particular between 0.5% and 5% by weight of active material relative to the total weight of the composition, more particularly between 1% and 4% by weight of active material relative to the total weight of the composition.

Preparation and Properties

The cosmetic composition according to this invention can be prepared by mixing the above essential and optional components using a conventional method. Preferably, the cosmetic composition is prepared by a method using low energy.

As described above, when the composition according to the invention does not comprise a surfactant as described in the corresponding section of this application, it corresponds to a micro-emulsion.

The “micro-emulsion” can be defined in two ways, in other words in a broad sense and in a more restricted sense. Specifically, in one case (“micro-emulsion in the restricted sense”), the micro-emulsion designates a single thermodynamically stable isotropic liquid phase containing a ternary system with three components comprising an oily component, an aqueous component and a surfactant, and in the other case (“micro-emulsion in the broad sense”), among typical thermodynamically unstable emulsion systems, the micro-emulsion also comprises emulsions with transparent or translucent appearance due to the smaller size of their particles (Satoshi Tomomasa, et al., Oil Chemistry, vol. 37, No. 11 (1988), p. 48-53). In this context, “micro-emulsion” designates a “micro-emulsion in the restricted sense”, in other words a single thermodynamically stable isotropic liquid phase.

Micro-emulsion designates a state of an O/W (oil-in-water) type micro-emulsion in which the oil is solubilized by micella, a W/E (water-in-oil) type micro-emulsion in which water is solubilized by inverse micella, or a bicontinuous micro-emulsion in which the number of associations of surfactant molecules is made infinite so that the aqueous phase and the oily phase both have a continuous structure.

The micro-emulsion may have a dispersed phase with a mean diameter by number equal to 300 nm or less, preferably 200 nm or less and more preferably 100 nm or less, as measured by laser granulometry.

The composition according to the invention may include any additional ingredient well known to an expert in the subject, such as preservatives or fillers.

We will now give concrete examples illustrating the invention, but that are in no way limitative.

In the examples, the temperature is ambient temperature (20° C.) expressed in degrees Celsius unless mentioned otherwise, and the pressure is atmospheric pressure, unless mentioned otherwise.

In the examples, quantities of the ingredients of the compositions are given as a % by weight relative to the total weight of the composition (% w/w).

### EXAMPLES

In all the examples, the compositions were prepared with the ingredients mentioned in the following table, using the protocol described below:

The ingredients of phase B are mixed and heated to 80 C;
The ingredients of phase A are mixed and heated to 80 C;
Under Rayneri stirring, phase B is poured slowly into phase A;
Phase C is added at about 50° C.

Furthermore, in the examples, the protocol when tackiness on drying is measured is as follows:

Evaluation of Tackiness

The degree of tackiness when drying is evaluated in vivo:
50 microliters of formula are deposited on the back of the hand and applied by a circular movement at one turn per second until penetration;
the tackiness is then evaluated by touching the deposit with 2 fingers.

A score is assigned to the tackiness, that varies from 1 (not tacky) to 5 (very tacky).

A score strictly less than 2 is considered to be only slightly tacky and cosmetically acceptable.

### Example 1: Preparation of a Comparative Reference Composition

The comparative reference composition is prepared with the ingredients mentioned in the following table, using the protocol described above.

TABLE 1

| | | Formula |
|---|---|---|
| | | (% w/w) |
| Phase | Ingredient | (comparative) |
| C | SODIUM HYDROXIDE | 0.07 |
| A | POLYSACCHARIDE | 5 |
| | (INCI name: biosaccharide gum-2) | |
| | (Rhamnosoft HP 1.5P by Solabia) | |
| A | Preservative | Qs |
| B | CETYL ALCOHOL | 1 |
| B | STEARYL ALCOHOL | 1 |
| B | HYDROGENATED ISOPARAFFIN (6-8 | 6 |
| | MOLES OF ISOBUTYLENE) | |
| C | CARBOXYVINYLIC POLYMER | 0.25 |
| | SYNTHETIZED IN THE ETHYL | |
| | ACETATE/CYCLOHEXANE MIXTURE | |
| | (carbomer) | |
| B | CYCLOHEXA DIMETHYLSILOXANE | 4 |
| | (VISCOSITY: 8 CST) | |
| A | WATER | Qs 100 |
| A | GLYCERINE | 10 |
| B | GLYCERYL MONO/DISTEARATE/ | 2.5 |
| | POLYETHYLENE GLYCOL STEARATE | |
| | MIXTURE (100 OE) | |
| | (INCI name: GLYCERYL STEARATE | |
| | (and) PEG-100 STEARATE) | |
| | (Arlacel 165 by Croda) | |
| B | POLYETHYLENE GLYCOL STEARATE | 2.5 |
| | (40 OE) | |
| | (Myrj 52P by Croda) | |
| B | POLYGLYCERYL-5 LAURATE | 5 |
| | (SUNSOFT A-121E-C ® by Taiyo Kagaku) | |
| | Tackiness when drying | 3.5 |

This comparative composition has a tackiness when drying equal to 3.5.

### Example 2: Preparation of Comparative Compositions (F1 to F3) and a Composition According to the Invention (F4)

The following compositions F1 to F4 were prepared with the ingredients mentioned in the following table, using the protocol described above:

TABLE 2

| | | F1 | F2 | F3 | F4 |
|---|---|---|---|---|---|
| | | (comparative) | (comparative) | (comparative) | (invention) |
| Phase | Ingredient | (% w/w) | (% w/w) | (% w/w) | (% w/w) |
| A | POLYSACCHARIDE | 5 | 5 | 5 | 5 |
| | (INCI name: | | | | |
| | biosaccharide gum-2) | | | | |
| | (Rhamnosoft HP 1.5P | | | | |
| | by Solabia) | | | | |
| A | Preservative | Qs | Qs | Qs | Qs |
| B | 2-OCTYLDODECAN-1-OL | 2 | 5 | 7 | 15 |
| A | WATER | Qs 100 | Qs 100 | Qs 100 | Qs 100 |
| A | GLYCERINE | 10 | 10 | 10 | 10 |
| B | GLYCERYL | 1 | 1 | 1 | 1 |
| | MONO/DISTEARATE/ | | | | |
| | POLYETHYLENE | | | | |
| | GLYCOL STEARATE | | | | |
| | MIXTURE (100 OE) | | | | |
| | (INCI name: | | | | |
| | GLYCERYL | | | | |
| | STEARATE (and) | | | | |
| | PEG-100 STEARATE) | | | | |
| | (Arlacel 165 by Croda) | | | | |
| B | POLYETHYLENE | 1 | 1 | 1 | 1 |
| | GLYCOL STEARATE | | | | |
| | (40 OE) | | | | |
| | (Myrj 52P by Croda) | | | | |
| B | POLYGLYCERYL-5 | 5 | 5 | 5 | 5 |
| | LAURATE | | | | |
| | (SUNSOFT A-121E- | | | | |
| | C ® by Taiyo Kagaku) | | | | |
| | Tackiness when | 4 | 2 | 2 | 1.5 |
| | drying | | | | |

The results show that only formula F4 according to the invention, i.e. having at least 5% of octyldodecanol, has much lower tackiness than comparative formulas F1 to F3; but also than the comparative reference composition in example 1.

### Example 3: Preparation of Compositions According to the Invention (C1 to C3)

The following compositions C1 to C3 were prepared with the ingredients mentioned in the following table, using the protocol described above:

TABLE 3

| | | C1 | C2 | C3 |
|---|---|---|---|---|
| Phase | Ingredient | (% w/w) | (% w/w) | (% w/w) |
| A | POLYSACCHARIDE | 5 | 5 | 5 |
| | (INCI name: biosacchande gum-2) | | | |
| | (Rhamnosoft HP 1.5P by Solabia) | | | |
| C | AMORPHOUS SILICA | 2 | 2 | 2 |
| | MICROSPHERES (5 μm) | | | |
| A | Preservative | Qs | Qs | Qs |
| B | ISOPROPYL MYRISTATE | 5 | | |
| B | REFINE DEODORIZED | | | 2 |
| | STABILIZED SHEA BUTTER | | | |
| B | ISONONYL ISONONANOATE | | 5 | 5 |
| B | 1,3 PROPANEDIOL DICAPRYLATE | 5 | 5 | 5 |
| B | ACRYLAMIDO-2-METHYL | 0.4 | 0.4 | 0.4 |
| | PROPANE SODIUM | | | |
| | SULFONATE/HYDROXY- | | | |
| | ETHYL ACRYLATE COPOLYMER | | | |
| | (INCI: HYDROXYETHYL | | | |
| | ACRYLATE/SODIUM | | | |
| | ACRYLOYLDIMETHYL | | | |
| | TAURATE COPOLYMER); | | | |
| | (SEPINOV EMT 10 by Seppic) | | | |
| B | AMPS/ETHOXYLATED STEARYL | 0.7 | 0.7 | 0.7 |
| | METHACRYLATE (25 EO) | | | |
| | COPOLYMER CROSS-LINKED | | | |
| | BY TRIMETHYLOLPROPANE | | | |
| | TRIACRYLATE (INCI: | | | |
| | AMMONIUM ACRYLOYLDI- | | | |
| | METHYLTAURATE/ | | | |
| | STEARETH-25 METHACRYLATE | | | |
| | CROSSPOLYMER) | | | |
| | (ARISTOFLEX HMS by Clariant) | | | |
| A | Ethanol | 3 | 3 | 3 |
| A | WATER | Qs 100 | Qs 100 | Qs 100 |
| A | GLYCERINE | 10 | 10 | 10 |
| B | GLYCERYL MONO/DISTEARATE/ | 1 | 1 | 1 |
| | POLYETHYLENE GLYCOL | | | |
| | STEARATE MIXTURE (100 OE) | | | |
| | (INCI name: GLYCERYL | | | |
| | STEARATE (and) | | | |
| | PEG-100 STEARATE) | | | |
| | (Arlacel 165 by Croda) | | | |
| B | POLYETHYLENE GLYCOL | 1 | 1 | 1 |
| | STEARATE (40 OE) | | | |
| | (Myrj 52P by Croda) | | | |
| B | POLYGLYCERYL-5 | 5 | 5 | 5 |
| | LAURATE | | | |
| | (SUNSOFT A-121E- | | | |
| | C ® by Taiyo Kagaku) | | | |
| | Tackiness when drying | 1.5 | 1.5 | 1.5 |

The results show that the formulas C1 to C3 according to the invention, i.e. comprising at least 10% of oils chosen from among isopropyl myristate, shea butter, isononyl isononanoate and propanediol dicaprylate, have low tackiness when drying.

### Example 4: Preparation of Compositions According to the Invention (C4 to C9)

The following compositions C4 to C9 according to the invention were prepared with the ingredients mentioned in the following table, using the protocol described above:

TABLE 4

| | C4 | C5 | C6 | C7 | C8 | C9 |
|---|---|---|---|---|---|---|
| Ingredient | (% p/p) | (% p/p) | (% p/p) | (% p/p) | (% p/p) | (% p/p) |
| POLYSACCHARIDE | 5 | 5 | 5 | 5 | 5 | 5 |
| (INCI name: biosaccharide gum-2) | | | | | | |
| (Rhamnosoft HP 1.5P by Solabia) | | | | | | |
| AMORPHOUS SILICA | 2 | 2 | 2 | 2 | 2 | 2 |
| MICROSPHERES (5 μm) | | | | | | |
| Preservative | Qs | Qs | Qs | Qs | Qs | Qs |
| ISOPROPYL MYRISTATE | 20 | | | | | |
| 2-OCTYLDODECAN-1-OL | | 10 | | | | |
| ISONONYL ISONONANOATE | | | 10 | 20 | 30 | |
| 1,3 PROPANEDIOL DICAPRYLATE | | | | | | 10 |
| ACRYLAMIDO-2-METHYL | 0.4 | 0.4 | 0.4 | 0.4 | 0.4 | 0.4 |
| PROPANE SODIUM SULFONATE/ | | | | | | |
| HYDROXYETHYL ACRYLATE | | | | | | |
| COPOLYMER | | | | | | |
| (INCI: HYDROXYETHYL | | | | | | |
| ACRYLATE/SODIUM | | | | | | |
| ACRYLOYLDIMETHYL | | | | | | |
| TAURATE COPOLYMER); | | | | | | |
| (SEPINOV EMT 10 by Seppic) | | | | | | |
| AMPS/ETHOXYLATED | 0.7 | 0.7 | 0.7 | 0.7 | 0.7 | 0.7 |
| STEARYL METHACRYLATE | | | | | | |
| (25 EO) COPOLYMER | | | | | | |
| CROSS-LINKED BY | | | | | | |
| TRIMETHYLOLPROPANE | | | | | | |
| TRIACRYLATE | | | | | | |
| (INCI: AMMONIUM | | | | | | |
| ACRYLOYLDIMETHYLTAURATE/ | | | | | | |
| STEARETH-25 METHACRYLATE | | | | | | |
| CROSSPOLYMER) | | | | | | |
| (ARISTOFLEX HMS by Clariant) | | | | | | |
| Ethanol | 3 | 3 | 3 | 3 | 3 | 3 |
| WATER | Qs 100 | Qs 100 | Qs 100 | Qs 100 | Qs 100 | Qs 100 |
| GLYCERINE | 10 | 10 | 10 | 10 | 10 | 10 |
| GLYCERYL MONO/DISTEARATE/ | 1 | 1 | 1 | 1 | 1 | 1 |
| POLYETHYLENE GLYCOL | | | | | | |
| STEARATE MIXTURE (100 OE) | | | | | | |
| (INCI name: GLYCERYL | | | | | | |
| STEARATE (and) PEG-100 | | | | | | |
| STEARATE) | | | | | | |
| (Arlacel 165 by Croda) | | | | | | |
| POLYETHYLENE GLYCOL | 1 | 1 | 1 | 1 | 1 | 1 |
| STEARATE (40 OE) | | | | | | |
| (Myrj 52P by Croda) | | | | | | |
| POLYGLYCERYL-5 LAURATE | 5 | 5 | 5 | 5 | 5 | 5 |
| (SUNSOFT A-121E-C ® by Taiyo | | | | | | |
| Kagaku) | | | | | | |
| Tackiness when drying | 1 | 1 | 1.5 | 1.5 | 1 | 1.5 |

The results show that the formulas C4 to C9 according to the invention, i.e. comprising at least 10% of oils chosen from among isopropyl myristate, octyldodecanol, isononyl isononanoate and propanediol dicaprylate, have low tackiness when drying.

### Example 5: Preparation of Compositions According to the Invention (C10 to C14)

The following compositions 010 to C14 according to the invention were prepared with the ingredients mentioned in the following table, using the protocol described above:

TABLE 5

| | C10 | C11 | C12 | C13 | C14 |
|---|---|---|---|---|---|
| | (% | (% | (% | (% | (% |
| Ingredient | w/w) | w/w) | w/w) | w/w) | w/w) |
| POLYSACCHARIDE | 5 | 5 | 5 | 5 | 5 |
| (INCI name: biosaccharide gum-2) | | | | | |
| (Rhamnosoft HP 1.5P by Solabia) | | | | | |
| AMORPHOUS SILICA MICROSPHERES (5 | 2 | 2 | 2 | 2 | 2 |
| μm) | | | | | |
| Preservative | Qs | Qs | Qs | Qs | Qs |
| C15-C16 BRANCHED ALKANES | 10 | 20 | 30 | | |
| (Emogreen L15 by Seppic) | | | | | |
| C17-C18 BRANCHED ALKANES | | | | 10 | |
| (Emogreen L19 by Seppic) | | | | | |
| C15-C19 BRANCHED ALKANES | | | | | 10 |
| (Emosmart L19 by Seppic) | | | | | |
| ACRYLAMIDO-2-METHYL PROPANE | 0.4 | 0.4 | 0.4 | 0.4 | 0.4 |
| SODIUM SULFONATE/HYDROXYETHYL | | | | | |
| ACRYLATE COPOLYMER | | | | | |
| (INCI: HYDROXYETHYL | | | | | |
| ACRYLATE/SODIUM | | | | | |
| ACRYLOYLDIMETHYL TAURATE | | | | | |
| COPOLYMER); | | | | | |
| (SEPINOV EMT 10 by Seppic) | | | | | |
| AMPS/ETHOXYLATED STEARYL | 0.7 | 0.7 | 0.7 | 0.7 | 0.7 |
| METHACRYLATE (25 EO) COPOLYMER | | | | | |
| CROSS-LINKED BY | | | | | |
| TRIMETHYLOLPROPANE TRIACRYLATE | | | | | |
| (INCI: AMMONIUM | | | | | |
| ACRYLOYLDIMETHYLTAURATE/ | | | | | |
| STEARETH-25 METHACRYLATE | | | | | |
| CROSSPOLYMER) | | | | | |
| (ARISTOFLEX HMS by Clariant) | | | | | |
| Ethanol | 3 | 3 | 3 | 3 | 3 |
| WATER | Qs 100 | Qs 100 | Qs 100 | Qs 100 | Qs 100 |
| GLYCERINE | 10 | 10 | 10 | 10 | 10 |
| GLYCERYL MONO/DISTEARATE/ | 1 | 1 | 1 | 1 | 1 |
| POLYETHYLENE GLYCOL STEARATE | | | | | |
| MIXTURE (100 OE) | | | | | |
| (INCI name: GLYCERYL STEARATE (and) | | | | | |
| PEG-100 STEARATE) | | | | | |
| (Arlacel 165 by Croda) | | | | | |
| POLYETHYLENE GLYCOL STEARATE (40 | 1 | 1 | 1 | 1 | 1 |
| OE) | | | | | |
| (Myrj 52P by Croda) | | | | | |
| POLYGLYCERYL-5 LAURATE | 5 | 5 | 5 | 5 | 5 |
| (SUNSOFT A-121E-C ® by Taiyo Kagaku) | | | | | |
| Tackiness when drying | 1 | 1 | 1 | 1.5 | 1.5 |

The results show that the formulas 010 to C14 according to the invention, i.e. comprising at least 10% of oils chosen from among C15-C19 branched alkanes, have low tackiness when drying.

### Example 6: Preparation of Comparative Compositions

The following compositions C15 to C17 were prepared with the ingredients mentioned in the following table, using the protocol described above.

TABLE 6

| | Formula | Formula | Formula |
|---|---|---|---|
| | Comparative | Comparative | Comparative |
| | C15 | C16 | C17 |
| Ingredient | (% w/w) | (% w/w) | (% w/w) |
| POLYSACCHARIDE | 5 | 5 | 5 |
| (INCI name: biosacchande gum-2) | | | |
| (Rhamnosoft HP 1.5P by Solabia) | | | |
| AMORPHOUS SILICA MICROSPHERES (5 μm) | 2 | 2 | 2 |
| Preservative | Qs | Qs | Qs |
| DIPENTAERYTHRITYL | 10 | 20 | 30 |
| PENTAISONONANOATE | | | |
| ACRYLAMIDO-2-METHYL PROPANE SODIUM | 0.4 | 0.4 | 0.4 |
| SULFONATE/HYDROXYETHYL ACRYLATE | | | |
| COPOLYMER | | | |
| (INCI: HYDROXYETHYL ACRYLATE/SODIUM | | | |
| ACRYLOYLDIMETHYL TAURATE | | | |
| COPOLYMER); | | | |
| (SEPINOV EMT 10 by Seppic) | | | |
| AMPS/ETHOXYLATED STEARYL | 0.7 | 0.7 | 0.7 |
| METHACRYLATE (25 EO) COPOLYMER | | | |
| CROSS-LINKED BY TRIMETHYLOLPROPANE | | | |
| TRIACRYLATE | | | |
| (INCI: AMMONIUM | | | |
| ACRYLOYLDIMETHYLTAURATE/STEARETH- | | | |
| 25 METHACRYLATE CROSSPOLYMER) | | | |
| (ARISTOFLEX HMS by Clariant) | | | |
| Ethanol | 3 | 3 | 3 |
| WATER | Qs 100 | Qs 100 | Qs 100 |
| GLYCERINE | 10 | 10 | 10 |
| GLYCERYL MONO/DISTEARATE/ | 1 | 1 | 1 |
| POLYETHYLENE GLYCOL STEARATE | | | |
| MIXTURE (100 OE) | | | |
| (INCI name: GLYCERYL STEARATE (and) PEG- | | | |
| 100 STEARATE) | | | |
| (Arlacel 165 by Croda) | | | |
| POLYETHYLENE GLYCOL STEARATE (40 OE) | 1 | 1 | 1 |
| (Myrj 52P by Croda) | | | |
| POLYGLYCERYL-5 LAURATE | 5 | 5 | 5 |
| (SUNSOFT A-121E-C ® by Taiyo Kagaku) | | | |
| Tackiness when drying | 2 | 2.5 | 3 |

The results show that these comparative formulas, i.e. including various quantities of dipentaerythrityl pentaisononanoate, have strong tackiness when drying.

### Example 7: Preparation of Compositions According to the Invention and Comparison with Formula C16

The following four compositions according to the invention, and the comparative composition C16, were prepared with the ingredients mentioned in the following table, using the protocol described above:

TABLE 7

| | | Formula | Formula | Formula | Formula |
|---|---|---|---|---|---|
| | | according | according | according | according |
| | Comparative | to | to | to | to |
| | formula | the | the | the | the |
| Ingredient | C16 | invention | invention | invention | invention |
| POLYSACCHARIDE | 5 | 5 | 5 | 5 | 5 |
| (INCI name: biosaccharide gum-2) | | | | | |
| (Rhamnosoft HP 1.5P by Solabia) | | | | | |
| AMORPHOUS SILICA MICROSPHERES (5 | 2 | 2 | 2 | 2 | 2 |
| μm) | | | | | |
| preservative | qs | qs | Qs | Qs | qs |
| DIPENTAERYTHRITYL | 20 | | | | |
| PENTAISONONANOATE | | | | | |
| Jojoba oil | | 20 | | | |
| Isononyl isononanoate | | | 20 | | |
| Isopropyl myristate | | | | 20 | |
| Mixture of C15-C19 branched alkanes | | | | | 20 |
| (Emogreen L15) | | | | | |
| ACRYLAMIDO-2-METHYL PROPANE SODIUM | 0.4 | 0.4 | 0.4 | 0.4 | 0.4 |
| SULFONATE/HYDROXYETHYL ACRYLATE | | | | | |
| COPOLYMER (SEPINOV EMT 10 by Seppic) | | | | | |
| AMPS/ETHOXYLATED STEARYL | 0.7 | 0.7 | 0.7 | 0.7 | 0.7 |
| METHACRYLATE (25 EO) COPOLYMER | | | | | |
| CROSS-LINKED BY TRIMETHYLOLPROPANE | | | | | |
| TRIACRYLATE (INCI: AMMONIUM | | | | | |
| ACRYLOYLDIMETHYLTAURATE/ | | | | | |
| STEARETH-25 METHACRYLATE | | | | | |
| CROSSPOLYMER)) | | | | | |
| (ARISTOFLEX HMS by Clariant) | | | | | |
| Ethanol | 3 | 3 | 3 | 3 | 3 |
| Water | Qs 100 | Qs 100 | Qs 100 | Qs 100 | Qs 100 |
| GLYCERINE | 10 | 10 | 10 | 10 | 10 |
| GLYCERYL MONO/DISTEARATE/ | 1 | 1 | 1 | 1 | 1 |
| POLYETHYLENE GLYCOL STEARATE | | | | | |
| MIXTURE (100 OE) | | | | | |
| (INCI name: GLYCERYL STEARATE (and) | | | | | |
| PEG-100 Stearate (Arlacel 165 by Croda) | | | | | |
| POLYETHYLENE GLYCOL STEARATE (40 | 1 | 1 | 1 | 1 | 1 |
| OE) | | | | | |
| (Myrj 52P by Croda) | | | | | |
| POLYGLYCERYL-5 LAURATE | 5 | 5 | 5 | 5 | 5 |
| (SUNSOFT A-121 E-C ® by Taiyo Kagaku) | | | | | |
| tackiness when drying | 2.5 | 1.5 | 1.5 | 1 | 1 |

The results show that only the formulas according to the invention, each including at least one oil according to the invention, have very low tackiness at the end of application. After 2 minutes 45 s, the tackiness of formula C16 is 2, while the tackiness of each of the formulas according to the invention is equal to 1.

Therefore the tackiness after drying is lower with formulas according to the invention, relative to the comparative formula C16.

### Example 8: Comparison of the Stability of Comparative Formulas (F9 to F13)

The following comparative formulas F9 to 13 were prepared as described above. Their composition is given in Tables below.

TABLE 8

| | F9 | F10 | F11 |
|---|---|---|---|
| | (com- | (com- | (com- |
| Ingredient | parative) | parative) | parative) |
| Biosaccharide gum-2 | 5 | 5 | 5 |
| (Rhamnosoft HP 1.5P by | | | |
| Solabia) | | | |
| (2.5% active material) | | | |
| AMORPHOUS SILICA | 3 | 3 | 3 |
| MICROSPHERES (5 μm) | | | |
| SPHERICAL CELLULOSE | 4.7 | 4.7 | 4.7 |
| BALLS (SIZE 4-7 μM) | | | |
| Preservative | Qs | Qs | Qs |
| ISONONYL | 18.54 | 18.54 | 18.54 |
| ISONONANOATE | | | |
| DIPENTAERYTHRITYL | 2.85 | 2.85 | 2.85 |
| PENTAISONONANOATE | | | |
| SODIUM ACRYLAMIDO-2- | 0.4 | 0.4 | 0.4 |
| METHYL PROPANE | | | |
| SULFONATE/ | | | |
| HYDROXYETHYLACRYLATE | | | |
| COPOLYMER IN POWDER | | | |
| FORM | | | |
| (Sepinov EMT 100 by Seppic) | | | |
| AMPS/ETHOXYLATED | 0.7 | 0.7 | 0.7 |
| STEARYL METHACRYLATE | | | |
| (25 EO) COPOLYMER | | | |
| CROSS-LINKED BY | | | |
| TRIMETHYLOLPROPANE | | | |
| TRIACRYLATE (TMPTA) | | | |
| (Aristoflex HMS by Clariant) | | | |
| Ethanol | 3 | 3 | 3 |
| WATER | Qs 100 | Qs 100 | Qs 100 |
| GLYCERIN | 10 | 10 | 10 |
| GLYCERYL STEARATE | 1 | 1 | 1 |
| (and) PEG-100 STEARATE | | | |
| (Arlacel 165 by Croda) | | | |
| PEG-40 STEARATE | 1 | 1 | 1 |
| (Myrj 52 P by Croda) | | | |
| PG-10 laurate | 5 | 2.5 | 1 |
| (DECAGLYCERYL | | | |
| MONOLAURATE) | | | |
| (DERMOFEEL G 10 L by Dr | | | |
| Straetmans) | | | |

For these formulas comprising 1%, 2.5% or 5% by weight of PG-10 laurate (F11, F10 and F9 respectively), a reduction in the stability and slip on application are observed.

Thus, PG-10 laurate does not confer the same effects as the fatty acid and polyglycerol ester comprising 5 to 9 glycerol patterns according to the invention.

TABLE 9

| | F12 | F13 |
|---|---|---|
| Ingredient | (comparative) | (comparative) |
| Biosaccharide gum-2 | 5 | 5 |
| (Rhamnosoft HP 1.5P by Solabia) | | |
| (2.5% active material) | | |
| AMORPHOUS SILICA MICROSPHERES (5 μm) | 3 | 3 |
| SPHERICAL CELLULOSE BALLS (SIZE 4-7μM) | 4.7 | 4.7 |
| Preservative | Qs | Qs |
| ISONONYL ISONONANOATE | 18.54 | 18.54 |
| DIPENTAERYTHRITYL PENTAISONONANOATE | 2.85 | 2.85 |
| SODIUM ACRYLAMIDO-2-METHYL PROPANE SULFONATE/ | 0.4 | 0.4 |
| HYDROXYETHYLACRYLATE COPOLYMER IN POWDER FORM | | |
| (Sepinov EMT 100 by Seppic) | | |
| AMPS/ETHOXYLATED STEARYL METHACRYLATE (25 EO) | 0.7 | 0.7 |
| COPOLYMER CROSS-LINKED BY TRIMETHYLOLPROPANE | | |
| TRIACRYLATE (TMPTA) | | |
| (Aristoflex HMS by Clariant) | | |
| Ethanol | 3 | 3 |
| WATER | Qs | Qs |
| | 100 | 100 |
| GLYCERIN | 10 | 10 |
| GLYCERYL STEARATE (and) PEG-100 STEARATE | 1 | 1 |
| (Arlacel 165 by Croda) | | |
| PEG-40 STEARATE | 1 | 1 |
| (Myrj 52 P by Croda) | | |
| POLYGLYCERYL-4 LAURATE (TEGO CARE PL 4 by Evonik) | 5 | 2.5 |

The formulas cannot be produced using PG-4 laurate: the incorporation of 2.5% or 5% by weight of PG-4 laurate, as in comparative formulas F12 and F13, leads to sedimentation and salting out: the formulas cannot be made.

Thus, PG-4 laurate does not confer the same effects as the ester of fatty acid and of polyglycerol comprising 5 to 9 glycerol patterns according to the invention.

### Example 9: Formula According to the Invention

The following formula according to the invention comprising a hydrophilic active agent is prepared.

TABLE 10

| | Formula |
|---|---|
| Ingredient | (invention) |
| Biosaccharide gum-2 | 7.5 |
| (Rhamnosoft HP 1.5P by Solabia) | |
| (2.5% active material) | |
| AMORPHOUS SILICA MICROSPHERES (5 μm) | 3 |
| SPHERICAL CELLULOSE BALLS (SIZE 4-7 μM) | 4.7 |
| Preservative | Qs |
| ISONONYL ISONONANOATE | 18.54 |
| DIPENTAERYTHRITYL PENTAISONONANOATE | 2.85 |
| SODIUM ACRYLAMIDO-2-METHYL PROPANE SULFONATE/ | 0.4 |
| HYDROXYETHYLACRYLATE COPOLYMER IN POWDER FORM | |
| (Sepinov EMT 100 by Seppic) | |
| AMMONIUM ACRYLOYLDIMETHYLTAURATE/STEARETH-25 | 0.7 |
| METHACRYLATE CROSSPOLYMER | |
| (Aristoflex HMS by Clariant) | |
| Ethanol | 3 |
| WATER | Qs 100 |
| GLYCERIN | 10 |
| GLYCERYL STEARATE (and) PEG-100 STEARATE | 1 |
| (Arlacel 165 by Croda) | |
| PEG-40 STEARATE | 1 |
| (Myrj 52 P by Croda) | |
| POLYGLYCERYL-5 LAURATE (SUNSOFT A-121E-C ® by Taiyo Kagaku) | 5 |
| Sodium hyaluronate | 0.5 |
| Hydroxypropyl tetrahydropyrantriol | 8.7 |
| (MEXORYL SCN ® of CHIMEX | (=3.045% |
| (35% active material) | active |
| | material) |

1. A composition comprising in a physiologically acceptable medium:
at least one polysaccharide containing rhamnose,
at least one polyol,
at least one ester of fatty acid and of polyglycerol comprising 5 to 9 glycerol patterns, and at least 10% by weight of at least one non-volatile hydrocarbon oil relative to the total weight of the composition, chosen from among C₁₀-C₂₆ monoalcohols; monoesters with the formula R₁COOR₂ wherein R₁ represents the remainder of a saturated or unsaturated, linear or branched or aromatic fatty acid comprising 4 to 40 carbon atoms, and R₂ represents a hydrocarbon chain containing 3 to 40 carbon atoms, provided that R₁+R₂ is greater than or equal to 17; the diesters comprising between 17 and 40 carbon atoms in total; plant hydrocarbon oils; linear or branched alkanes having 15 to 19 carbon atoms; and mixtures thereof.

2. The composition according to claim 1, wherein the polysaccharide comprises rhamnose amount varying from 10% to 100% by weight relative to the total weight of polysaccharide.

3. The composition according to claim 1, wherein the repetitive elements that form the polysaccharide comprise at least components with general formula I: 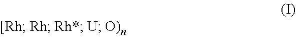
wherein Rh is a rhamnose molecule, Rh* is a rhamnose molecule fixed in a branched manner, O is a molecule of a hexosidic or pentosidic sugar, U is a molecule of uronic acid and n is between 1 and 100.

4. A composition according to claim 1, wherein the repetitive elements forming the polysaccharide are composed of the sequence with general formula II: 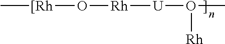
wherein Rh is a rhamnose molecule, O is a hexosidic or pentosidic sugar molecule, U is a uronic acid molecule and the rhamnose branch onto the ose O consists of an osidic bond (1→2) or (1→3).

5. The composition according to claim 1, wherein the repetitive elements forming the polysaccharide are composed of the sequence with general formula III: 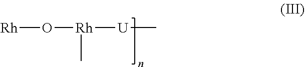
wherein Rh is a rhamnose molecule, 0 is a hexosidic or pentosidic sugar molecule, U is a uronic acid molecule and rhamnose is branched onto the rhamnose by an osidic bond (1→3).

6. A composition according to claim 1, wherein the polysaccharide is a polymer with a branched structure, with a molecular weight of the order of 50 000 daltons, and having a saccharidic sequence comprising three molecules of rhamnose (I, III, VI), two molecules of galactose (II, V) and one molecule of glucuronic acid (IV), said sequence having the following formula:

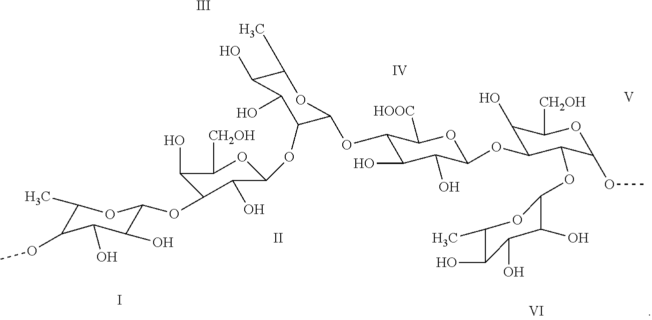

7. A composition according to claim 1, wherein the ester of fatty acid and of polyglycerol is formed from at least one acid comprising an alkyl or alkenyl chain containing from 12 to 20 carbon atoms and 5 to 9 glycerol patterns, preferably from 5 to 6 glycerol patterns.

8. A composition according to claim 1, wherein the ester of fatty acid and of polyglycerol is present in the composition according to the invention in a content ranging from 1% to 10% by weight relative to the total weight of the composition.

9. A composition according to claim 1 the polyol is chosen from among glycerine and its derivatives, and glycols and their derivatives, and mixtures thereof.

10. A composition according to claim 1 the polyol is present in a content ranging from 2% to 30% by weight, relative to the total weight of the composition.

11. A composition according to claim 1, wherein the oil is a fatty alcohol comprising 10 to 24 carbon atoms.

12. A composition according to claim 1, wherein the oil is octyldodecanol, present between 10% and 30% by weight of the total weight of the composition, preferably between 10% and 20% by weight.

13. A composition according to claim 1, wherein the oil is chosen from among Purcellin oil, isononyl isononanoate, C₁₂ to C₁₅ alcohol benzoate, 2-ethyl hexyl palmitate, octyldodecyl neopentanoate, octyl-2 dodecyl stearate, octyl-2-dodecyl erucate, isostearyl isostearate, octyl-2 dodecyl benzoate, octanoates, decanoates or ricinoleates of alcohols or of polyalcohols, isopropyl myristate, isopropyl palmitate, butyl stearate, hexyl laurate, 2-ethyl-hexyl palmitate, 2-hexyl-decyl laurate, 2-octyl-decyl palmitate and 2-octyldodecyl myristate.

14. A composition according to claim 1, wherein the oil is isononyl isononanoate; and/or isopropyl myristate.

15. A composition according to claim 1, wherein the oil is 1,3-propanediol dicaprylate.

16. A composition according to claim 1, wherein the oil is a plant hydrocarbon oil.

17. A composition according to claim 1, wherein the oil is a mixture of alkanes.

18. A composition according to claim 1, wherein it comprises at least one surfactant that is a fatty acid and polyethylene glycol ester.

19. A composition according to claim 1, which comprises at least one hydrophilic active agent.

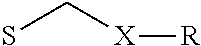

20. A cosmetic method for cleansing keratin materials comprising the application of a composition according to claim 1 onto said keratin materials.

21. A cosmetic method for making the skin more supple, particularly the stratum corneum, comprising applying a composition according to claim 1 onto the skin.

22. A composition comprising, in a physiologically acceptable medium:
at least one polysaccharide comprising rhamnose,
at least at least one hydrophilic active agent, 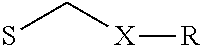
and
optionally at least 10% by weight relative to the total weight of composition, of at least one non-volatile hydrocarbon oil chosen from among:
C₁₀-C₂₆ monoalcohols,
monoesters with formula R₁COOR₂ wherein R₁ is the remainder of a linear or branched or aromatic fatty acid comprising from 4 to 40 saturated or unsaturated carbon atoms and R₂ is a hydrocarbon chain, with the condition that R₁+R₂ is greater than or equal to 17;
diesters comprising between 17 and 40 carbon atoms in total,
vegetable hydrocarbon oils;
linear or branched alkanes having from 15 to 19 carbon atoms; and
mixtures thereof.
